(19) United States
(12) Patent Application Publication
(10) Pub. No.: US 2015/0190388 A1
(43) Pub. Date: Jul. 9, 2015

(54) PHARMACEUTICAL COMPOSITION OF MOXIFLOXACIN HYDROCHLORIDE AND PREPARATION METHOD

(71) Applicant: RIVOPHARM SA, Manno (CH)

(72) Inventors: Piero Poli, Lugano (CH); Michela Carcano, Maslianico (IT); Gabriele Vecchi, Como (IT)

(73) Assignee: RIVOPHARM SA, Manno (CH)

(21) Appl. No.: 14/409,576

(22) PCT Filed: Jun. 21, 2013
(86) PCT No.: PCT/EP2013/063043

§ 371 Date: Dec. 19, 2014

(30) Foreign Application Priority Data
Jun. 22, 2012 (FR) 1255959

(51) Int. Cl. A61K 31/4709; A61K 47/12; A61K 47/32; A61K 47/02; A61K 47/38; A61K 47/36

(52) U.S. Cl. CPC A61K 31/4709; A61K 47/38; A61K 47/36; A61K 47/32; A61K 47/02; A61K 47/12

(57) ABSTRACT

The subject of the present invention is a method for preparing a pharmaceutical moxifloxacin hydrochloride composition in solid form, which comprises mixing a moxifloxacin hydrochloride having a water content of less than 2% by weight with one or more pharmaceutically acceptable excipients. The subject of the invention is also a pharmaceutical composition obtainable by this method.

### FIELD OF THE INVENTION

The subject of the invention is a pharmaceutical moxifloxacin hydrochloride composition and a method for the preparation thereof.

### TECHNICAL BACKGROUND OF THE INVENTION

Moxifloxacin (International Non-Proprietary Name—INN) and also the method for the synthesis thereof have been described in patent EP 550 903. Moxifloxacin corresponds to 1-cyclopropyl-6-fluoro-1,4-dihydro-8-methoxy-7-[(4aS,7aS)-octahydro-6H-pyrrolo[3,4-b]pyridin-6-yl]-4-oxo-3-quinolinecarbolic acid and has the following chemical formula:

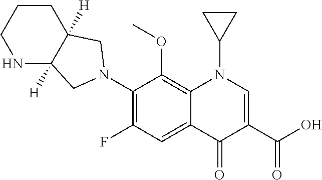 [SMILES: COc1c(N2C[C@@H]3CCCN[C@@H]3C2)c(F)cc2c(=O)c(C(=O)O)cn(C3CC3)c12]

Moxifloxacin is a broad-spectrum antibacterial agent of the fluoroquinolone class. Its hydrochloride form is the active ingredient of the proprietary specialty product Avelox® (called Izilox® in France). Avelox® is sold, inter alia, in the form of film-coated tablets and is indicated for the treatment of certain gynaecological infections and of respiratory infections such as acute bacterial sinusitis, acute bronchitis and pneumonia. Moxifloxacin hydrochloride exists in various crystalline polymorphic forms corresponding either to anhydrous forms or to hydrate forms. By way of example, U.S. Pat. No. 5,849,752 describes two crystalline forms: a first crystalline form called form I corresponding to an anhydrous moxifloxacin hydrochloride (moxifloxacin.HCl) and a second crystalline form called form II corresponding to a moxifloxacin hydrochloride monohydrate (moxifloxacin.HCl.H₂O).

Other anhydrous polymorphs of moxifloxacin hydrochloride are described in EP 2 089 388, EP 1 685 130 and EP 1 615 645. Patent applications EP 2 342 204 and EP 2 342 204 describe, for their part, monohydrate polymorphic forms.

The polymorphic forms of moxifloxacin hydrochloride can exhibit distinct stability and pharmacological properties, in particular in terms of bioavailability. In this respect, one should remind that the hydrate forms are generally described as having a lower dissolution rate than the anhydrous forms.

Since moxifloxacin hydrochloride exists in various polymorphic forms, it is therefore essential to use formulation methods which enables to control its polymorphism in the final pharmaceutical composition.

U.S. Pat. No. 6,610,327 describes a method for preparing a pharmaceutical moxifloxacin composition by wet granulation. Nevertheless, U.S. Pat. No. 6,610,327 is not interested in the problem relating to the polymorphism of moxifloxacin.

International application WO 2010/041100 provides a method for obtaining a stable pharmaceutical composition of anhydrous moxifloxacin hydrochloride. This method comprises a step of wet granulation using, as wetting agent, an organic solvent such as dichloromethane or ethanol.

Finally, international application WO 2009/135646 provides a formulation method comprising the mixing of a moxifloxacin hydrochloride with at least a diluent which has a low water content, and then the direct compaction or the dry granulation of the mixture obtained.

There is still at the current time a need for methods for formulating moxifloxacin hydrochloride which enables to maintain its starting polymorphic form.

### SUMMARY OF THE INVENTION

A subject of the present invention is a method for preparing a pharmaceutical moxifloxacin hydrochloride composition in solid form, comprising the steps consisting in:
a) providing a moxifloxacin hydrochloride having a water content of less than 2% by weight,
b) mixing the moxifloxacin hydrochloride with one or more pharmaceutically acceptable excipients,
c) shaping the mixture obtained in step b) so as to obtain a solid form, preferably a tablet,
d) subjecting the solid form obtained in step c) to a temperature ranging from 35° C. to 100° C., and
e) coating the solid form with a coating agent.

Preferably, the moxifloxacin hydrochloride of step a) is in anhydrous form.

In certain embodiments, step d) is carried out in such a way that the water content of the solid form is less than 2% by weight, preferably less than 1.5%, or even less than 1.0%.

In certain embodiments, the method is characterized by one or several (1, 2, 3, 4, 5, 6 or 7) of the following features:
the resulting final solid form has a water content of less than 2% by weight,
step e) is carried out directly at the end of step d),
step d) is carried out at a temperature of 45° C. to 85° C. for a period ranging from 10 min to 24 h,
in step b), moxifloxacin hydrochloride is mixed at least with a diluent and a binder,
in step b), moxifloxacin hydrochloride is mixed at least with a diluent and a binder and with at least an excipient selected from the group consisting of a disintegrant, a lubricant, a flow agent, and mixtures thereof,
step c) comprises the substeps consisting in:
   c1) carrying out a dry granulation on the mixture obtained in step b) so as to obtain granules and, optionally, sieving the resulting granules,
   c2) mixing the granules obtained in substep c1) with one or more excipients, preferably selected from the group consisting of a disintegrant, a lubricant and a flow agent, and
   c3) shaping the granules obtained at the end of substep c2) in the form of (a) tablet(s),
in step c), the solid forms are obtained by direct compression or by dry granulation.

An additional subject according to the invention is a pharmaceutical composition obtainable, or obtained, by the method above, and also the use of said composition for the treatment of an infectious disease in a patient.

### FIGURES

FIG. 1 provides an example of implementation of the method according to the invention.

FIG. 2 shows the X-ray powder diffraction (XRPD) spectra of a moxifloxacin hydrochloride in anhydrous form (curve 1), after treatment for several hours at a relative humidity of approximately 40% (curve 3) and after drying (curve 2). x-axis: 2-theta angle) (28° and y-axis: intensity. These spectra show that the anhydrous moxifloxacin hydrochloride is hygroscopic and has a tendency to convert into hydrate in the presence of a relative humidity of approximately 40%. It is possible to reconvert the hydrate formed into an anhydrous polymorphic form by drying.

FIG. 3 shows the X-ray diffraction spectra of the tablets according to the present invention after exposure to a temperature of 25° C. and a relative humidity of 60% (curve 1) and after exposure to a temperature of 40° C. and a relative humidity of 75% (curve 2) for 1 month. Curve 3 corresponds to the anhydrous moxifloxacin hydrochloride spectrum and curve 4 corresponds to a placebo tablet spectrum (without moxifloxacin). x-axis: 2-theta)(28° angle and y-axis: intensity.

These spectra show that, after storage of the tablets in the presence of a high temperature and a high relative humidity, the anhydrous moxifloxacin hydrochloride did not convert into a hydrate form, which attests to the stability of the composition according to the invention.

### DETAILED DESCRIPTION OF THE INVENTION

### Method for Preparing a Pharmaceutical Moxifloxacin Composition

A subject of the present invention is a method for preparing a pharmaceutical moxifloxacin hydrochloride composition. The method according to the invention relates more specifically to the obtaining of film-coated solid pharmaceutical forms such as film-coated tablets, preferably intended for oral administration. As is shown in Example 1 of the present application, the hydrochloride forms of moxifloxacin can exhibit low stability even in the presence of a relative humidity of 40%. A variation in their water content can result in their conversion into other polymorphic forms. In order to prevent the conversion of the starting form of moxifloxacin hydrochloride into another polymorphic form during the formulating method, it is preferable for the starting hydrochloride to have a water content of less than 2% by weight.

The applicant has also shown that it is possible to improve the stability of the final film-coated pharmaceutical forms and to be sure that said pharmaceutical forms contain essentially an anhydrous moxifloxacin hydrochloride by subjecting the solid forms obtained after shaping to a heating step before they are film-coated. This heating step is generally carried out at a temperature ranging from 35° C. to 100° C., and enables to control the water content of the tablets.

Thus, the method for preparing a pharmaceutical moxifloxacin hydrochloride composition in solid form according to the invention is characterized in that it comprises the steps consisting in:
a) providing a moxifloxacin hydrochloride having a water content of less than 2% by weight,
b) mixing the moxifloxacin hydrochloride with one or more pharmaceutically acceptable excipients,
c) shaping the mixture obtained in step b) so as to obtain a solid form, preferably a tablet, and
d) subjecting the solid form obtained in step c) to a temperature ranging from 35° C. to 100° C., and
e) coating the solid form with a coating agent.

This method enables to obtain a pharmaceutical composition of moxifloxacin hydrochloride which is stable owing to its low water content. Preferably, the water content of the final pharmaceutical composition is less than 2% by weight. As shown by Example 3, such a composition is particularly stable with respect to humidity since conversion of the anhydrous moxifloxacin hydrochloride to other polymorphic forms is not observed during storage. Without wishing to be bound by any theory, the applicant is of the opinion that a small difference between the initial water content of the moxifloxacin hydrochloride and the overall water content of the tablet enables to limit the migrations of water molecules within the formulation matrix and therefore the conversion of the starting moxifloxacin hydrochloride to other polymorphic forms during the storage of the final pharmaceutical forms.

The term “a solid form” should be interpreted generically as meaning that the method according to the invention enables to obtain one or more identical solid forms. For the purposes of the invention, the water content of a compound is obtained by dividing the amount by weight of water associated with said compound (for example by hygroscopy) by the total weight of said compound and by multiplying the obtained quotient by 100 so as to obtain a percentage.

The moxifloxacin hydrochloride used for carrying out the method according to the invention corresponds either to a specific polymorphic form or to a mixture of polymorphic forms. Preferably, the mixture is enriched with a polymorphic form of moxifloxacin hydrochloride: it thus comprises at least 90% by weight, preferably at least 95% by weight, even more preferably at least 97% by weight of a specific polymorphic form of moxifloxacin hydrochloride.

In one preferred embodiment, the moxifloxacin hydrochloride is anhydrous. For the purpose of the invention, the term “an anhydrous moxifloxacin hydrochloride” denotes a moxifloxacin hydrochloride substance of which the predominant crystalline form of which it is composed does not comprise water molecules inserted into its crystalline network. Nevertheless, the anhydrous moxifloxacin hydrochloride may contain a low amount of residual water. Without wishing to be bound by any theory, the applicant thinks that these water molecules can become absorbed at the surface of the crystal or become lodged in the channels formed by the crystals. Thus, the anhydrous moxifloxacin hydrochloride according to the invention can comprise a water content of less than 2% by weight, preferably less than 1.5% by weight. A water content of less than 1.5% by weight encompasses a water content of less than 1.2%, a content of less than 1.0%, a content of less than 0.8%, a water content of less than 0.6%, a water content of less than 0.4% and a water content of less than 0.3% by weight.

The anhydrous moxifloxacin hydrochloride according to the invention can correspond to any one of the anhydrous polymorphic forms described in the prior art. In particular, it can be one of the anhydrous crystalline forms described in EP 2 089 388, EP 1 685 130, US 2010/0152229 and EP 1 615 645, and also mixtures thereof.

For example, the predominant polymorphic form of the moxifloxacin hydrochloride is an anhydrous moxifloxacin monohydrochloride crystalline form. This anhydrous moxifloxacin monohydrochloride may have an X-ray diffraction spectrum expressed in terms of 28 angles comprising the following 28 angle diffraction peaks: 7.1±0.2, 8.8±0.2, 13.1±0.2, 13.9±0.2, 16.6±0.2, 17.7±0.2 and 22.1±0.02.

The pharmaceutical excipients that are of use for carrying out step b) comprise in particular diluents, binders, disintegrant agents, lubricant agents and flow agents. Other excipients can also be added, such as colourants and flavourings. In one preferred embodiment, in step b) of the method, the moxifloxacin hydrochloride is mixed at least with a diluent and a binder, and optionally with at least an excipient selected from a flow agent, a lubricant and a disintegrant agent.

The excipients to be used for carrying out step b) and also, possibly, for another step of the method, vary depending on the shaping method used (step c) of the method) and on the desired active ingredient release profile.

Reference may be made to the reference book “Handbook of Pharmaceutical Excipients”, American Pharmaceutical Association (Pharmaceutical Press; 6th revised edition, 2009) which describes a large number of excipients in relation to the formulation method and the desired profile of release for the active ingredient. The preferred excipients for the purposes of the invention are described below in the present description.

During step d), the dosage forms are generally heated at a temperature ranging from 35° C. to 100° C., preferably from 50° C. to 100° C. Preferably, the solid forms obtained at the end of step d) have a water content of less than 2% by weight.

A heating temperature included in a range of 35° C. to 100° C. encompasses a heating temperature ranging from 40° C. to 90° C., from 40° C. to 85° C., from 45° C. to 80° C., from 45° C. to 85° C., from 50° C. to 90° C. or else from 60° C. to 85° C. In some embodiments, during step d), the dosage forms can be heated at a temperature ranging from 35° C. to 80° C., which encompasses heating from 35° C. to 40° C., from 40° C. to 45° C., from 45° C. to 50° C., from 50° C. to 55° C., from 55° C. to 60° C., from 60° C. to 65° C., from 65° C. 70° C., from 70° C. to 75° C. and of 80° C. The solid forms may be subjected to a temperature ranging from 35° C. to 65° C. or even to a temperature ranging from 40° C. to 60° C.

The duration of step d) is variable and depends on the means used to carry it out. Generally, the duration of step d) varies from a few minutes to a few hours, or even a few days. Step d) can last from a few minutes, for example 10 min, to a few hours, for example, 24 h.

In certain embodiments, step d) can last from 1 min to 8 h, typically from 5 min to 2 h. In certain embodiments, step d) lasts from 5 min to 1 h, which encompasses a duration of 5 min to 10 min, from 10 min to 15 min, from 15 min to 20 min, from 20 min to 30 min, from 30 min to 40 min, from 40 min to 50 min, from 50 min to 60 min, and from 10 min to 45 min. For example, step d) can have a duration ranging from 15 min to 30 min.

In other embodiments, step d) can last a few hours, for example from 2 h to 24 h, which encompasses a duration of 2 h to 5 h, 5 h to 10 h, from 10 h to 18 h and from 18 h to 24 h. For example, step d) is carried out at a temperature of 45° C. to 100° C., preferably 45° C. to 85° C., for a time ranging from 10 min to 24 h.

Step d) makes it possible to adjust the water content of the solid forms. For example, it can enable to decrease the water content to a value of less than 2% by weight, preferably less than 1.5% by weight, or even less than 1% and even less than 0.5%.

Step d) can also enable to eliminate the crystallization water molecules, thus making it possible to convert the moxifloxacin.HCl hydrates possibly present into anhydrous moxifloxacin hydrochloride.

The conditions for carrying out step d) can therefore be adjusted so as to obtain such a result. It goes without saying that the heating time and temperature of step d) depend on the water content of the solid forms. By way of example, if the solid forms comprise a percentage of water of at least 4%, the solid form can be subjected to a temperature of between 70° C.-100° C., typically 80° C., for a time of approximately 2 h-24 h, in order to obtain a stable final solid form comprising essentially anhydrous moxifloxacin.HCl. If the solid form comprises a water content close to 2%, heating the solid form at approximately 50° C.-70° C., for example at approximately 65° C., for 5-30 min may be sufficient.

Step d) can be carried out by any means known to those skilled in the art. Step d) can be, for example, carried out in an oven, on a fluidized bed or directly in the coating turbine. When step d) is carried out in an oven, the heating temperature corresponds to the temperature of the atmosphere of the oven. When step d) is carried out on a fluidized bed or in a coating turbine, the heating temperature corresponds to the temperature of the intake air.

The film-coating step e) is carried out at the end of step d). Steps d) and e) are therefore consecutive. Preferably, step e) is carried out directly at the end of step d).

Step e) can be carried out according to the conventional film-coating methods described in the prior art. By way of example, this step can be carried out by spraying the coating agent in solution or in suspension in a solvent (for example a C1-05 alcohol, such as ethanol) or in an aqueous or aqueous-alcoholic solution at the surface of the solid forms. This spraying may be followed by a drying step in order to remove the possible traces of solvent. Typically, the coating step can be carried out in a coating turbine. Notably, the film-coating step e) does not significantly modify the water content of the solid form.

Moreover, steps b) and c) of the method are preferably carried out under dry conditions, i.e. in the absence of solvent or of wetting agent, in particular in the absence of water. Under these conditions, step b) leads to a mixture of moxifloxacin hydrochloride and excipients which are in the form of a dry powder. This dry powder can be shaped, for example, by direct compression or by dry granulation.

It is not necessary, in order to obtain a stable final composition comprising moxifloxacin hydrochloride in essentially anhydrous form, to use, during the method, excipients which have a low water content or to control the level of ambient hygrometry, since the heating step d) can enable to remove both the water present in the excipients by hygroscopy and/or the crystallization water molecules possibly associated with the moxifloxacin molecules. Nevertheless, in certain embodiments of the method according to the invention, step b) and/or c) is/are carried out in the presence of a hygrometry degree of less than 45%, preferably from 20% to 40%. In specific embodiments, steps b) and c) are carried out in the presence of a relative humidity (RH) of less than 40%.

In other embodiments, one may use, in the steps preceding the coating step e), of excipients which have a low overall water content, generally less than 6% by weight, or less than 4% by weight and even less than 2% by weight. The overall water content of the excipients can be calculated by adding the water contents of the excipients used in the steps preceding the coating step e), each water content being weighted by the amount by weight of the corresponding excipient, and by dividing the sum obtained by the total weight of the excipients.

The water contents of the moxifloxacin hydrochloride and of the excipients can be determined using any one of the analytical methods described in the prior art. For example, one may use of the Karl Fisher titration method as described in the European Pharmacopoeia Chapter 2.5.12 (Ph. Eur. 2.5.12) using the Metrohm 7012 KF Titrino® titrator.

In certain cases, it may be advantageous not to integrate all the excipients into the mixture formed in step b). A fraction of the excipients can be kept back so as to be subsequently integrated into the method, preferably during the shaping step c). During the shaping step c), one or more excipients selected from a disintegrant, a lubricant and a flow agent can be added.

The addition of a lubricant and/or of a flow agent during step c) enables to facilitate the shaping into tablets and to improve the pharmacotechnical properties thereof. The addition of the disintegrant both in step b) and step c) of the method can enable to improve the active ingredient release properties in the final pharmaceutical form.

Thus, in certain embodiments of the method according to the invention, the shaping step c) comprises the substeps consisting in:
c1) carrying out a dry granulation of the mixture obtained in step c) so as to obtain granules,
c2) mixing the granules obtained in substep c1) with one or more excipients, preferably selected from the group consisting of a disintegrant, a lubricant and a flow agent, and
c3) preparing tablets from the granules obtained in substep c2), preferably by compression.

The granulation step c1) generally comprises the compacting of the mixture obtained in step b) so as to obtain slugs which are then crushed and sieved in order to obtain granules. The compacting can be carried out using a roll compactor.

Advantageously, in step c2), the granules can be mixed at least with a disintegrant, a lubricant and a flow agent.

Step c3) can be carried out using a tablet press.

In one particular embodiment, the method according to the invention is characterized in that:
in step b), the moxifloxacin hydrochloride is mixed at least with a diluent, a disintegrant, a binder, a flow agent and a lubricant, and
in substep c2), the granules are mixed at least with a disintegrant, a lubricant and a flow agent.

In one preferred embodiment, the amounts of excipients and of moxifloxacin hydrochloride used during the method enables to obtain a final pharmaceutical composition comprising:
from 50% to 70% by weight, preferably from 55% to 65% by weight, of anhydrous moxifloxacin hydrochloride,
from 1% to 10%, preferably from 4% to 8% by weight, of disintegrant,
from 1% to 6%, preferably from 2% to 4% by weight, of binder,
from 10% to 30%, preferably from 18% to 25% by weight, of diluent,
from 0.5% to 4%, preferably from 2.5% to 3.5% by weight, of lubricant,
from 0.1% to 3%, preferably from 1% to 2% by weight, of flow agent, and
from 1% to 5%, preferably from 2.5% to 3.5%, of a coating agent.

The percentages by weight are expressed relative to the total weight of the composition. Advantageously, the water content of the final pharmaceutical composition is less than 2% by weight, preferably less than 1.5% by weight.

It goes without saying that the pharmaceutical composition may comprise additional excipients, such as colourants or flavourings.

The disintegrant, the flow agent and the lubricant of step b) may be identical to or different from those of step c2).

For carrying out the method according to the invention, those skilled in the art may refer to reference books such as Remington: The Science and Practice of Pharmacy (Lippincott Williams & Wilkins; Twenty first Edition, 2005) and may use commercially available devices and raw materials. It goes without saying that the method according to the invention may comprise additional steps to those previously described, such as steps for drying one or more of the excipients introduced in steps b) and/or c) so as to lower their water content or steps for determining the water content of the excipients and of the moxifloxacin hydrochloride before the use thereof. Moreover, the method according to the invention may comprise a step for determining the water content in the solid form before the implementation of step d) in order to adjust the heating time and temperature of this step.

### Excipients Suitable for Implementing the Method According to the Invention

Various types of excipients can be used in the various embodiments of the method according to the invention. The preferred excipients are selected from diluents, binders, lubricants, flow agents and disintegrants.

For the purposes of the present invention, a diluent may be one or more compounds capable of densifying the active ingredient so as to obtain the desired mass. The diluents encompass inorganic phosphates, for example dibasic calcium phosphate, sugars such as lactose hydrate or anhydrous lactose, mannitol, cellulose and its derivatives, and also starches. By way of example, a diluent suitable for implementing the method according to the invention can be selected from microcrystalline cellulose, pregelatinized starch, dibasic calcium phosphate, and mixtures thereof.

The binder may be one or more compounds capable of improving the aggregation of the active ingredient with the diluent. By way of example, one may use hydroxypropyl cellulose, hydroxypropylmethyl cellulose, povidone (polyvinylpyrrolidone) and N-vinyl-2-pyrrolidone/vinyl acetate copolymers (copovidone).

The lubricant may be one or more compounds capable of preventing the problems associated with the preparation of dry forms, such as the problems of sticking and/or gripping which occur in the machines during compression or filling. The preferred lubricants are fatty acids or fatty acid derivatives, such as calcium stearate, glyceryl monostearate, glyceryl palmitostearate, magnesium stearate, sodium lauryl sulphate, zinc stearate or stearic acid, polyalkylene glycols, in particular polyethylene glycol, sodium benzoate or talc. The preferred lubricants according to the invention are talc, stearate salts, and mixtures thereof. The flow agent optionally used in the method according to the invention may be selected from compounds which contain silicon, for example anhydrous colloidal silica or precipitated silica.

The disintegrant may be one or more compounds selected from crosslinked polyvinylpyrrolidone (crospovidone), crosslinked carboxymethyl cellulose (such as sodium croscarmellose) or non-crosslinked carboxymethyl cellulose, and sodium starch glycolate. Finally, the coating agent may comprise a cellulose derivative, such as a methyl cellulose, a hydroxypropylmethyl cellulose (HPMC) and a hydroxymethyl cellulose, a povidone, a polyethylene glycol, a wax or else a polyvinyl alcohol. It may also contain a colourant, such as an iron oxide or an aluminium oxide, an opacifier and/or a plasticizer. The coating agent may correspond to a commercial composition, such as the Opadry II® composition sold by Colorcon.

In one particular embodiment, the excipients are selected in such a way that the final pharmaceutical composition comprises:
a disintegrant selected from the group consisting of a sodium croscarmellose, a crospovidone, a hydroxypropyl cellulose, which is preferably weakly crosslinked, a sodium starch glycolate, and mixtures thereof,
a binder selected from the group consisting of a copovidone, a povidone, a hydroxypropyl cellulose, a hydroxypropylmethyl cellulose, and mixtures thereof,
a diluent selected from the group consisting of a starch, a pregelatinized starch, a microcrystalline cellulose, lactose monohydrate, a sodium phosphate, preferably dibasic sodium phosphate, and mixtures thereof,
a lubricant selected from the group consisting of stearate salts, in particular magnesium stearate, talc, and mixtures thereof,
a flow agent selected from a silicon compound, preferably a colloidal silica, and
a coating agent selected from coating agents based on polyvinyl alcohol, on HPMC or on povidone.

In another particular embodiment, the excipients are selected in such a way that the final pharmaceutical composition comprises:
a disintegrant selected from the group consisting of a sodium croscarmellose, a crospovidone, a hydroxypropyl cellulose, which is preferably sparingly crosslinked, and mixtures thereof,
a binder selected from the group consisting of a copovidone, a povidone, a hydroxypropyl cellulose, a hydroxypropylmethyl cellulose (HPMC), and mixtures thereof,
a diluent selected from the group consisting of a starch, a pregelatinized starch, a microcrystalline cellulose, lactose monohydrate, and mixtures thereof,
a lubricant selected from the group consisting of stearate salts, in particular magnesium stearate, talc, and mixtures thereof,
a flow agent selected from a silicon compound, preferably a colloidal silica, and
a coating agent selected from coating agents based on polyvinyl alcohol, on HPMC or on povidone.

In one particular embodiment, the pharmaceutical composition obtained at the end of the method comprises a copovidone, a sodium croscarmellose, a pregelatinized starch, a microcrystalline cellulose, colloidal silica, magnesium stearate and a coating agent based on polyvinyl alcohol.

As specified above, the moxifloxacin hydrochloride is preferably an anhydrous polymorphic form or a mixture of polymorphic forms enriched with an anhydrous polymorphic form. The amount of moxifloxacin present in the final dosage form corresponds to a pharmaceutically effective amount. This amount may be between 50 mg and 1 g. Preferably, the amount of moxifloxacin per dosage unit is between 300 mg and 600 mg. It is typically approximately 400 mg.

Pharmaceutical Compositions and Uses Thereof

An additional subject of the present invention is a pharmaceutical composition obtainable, or obtained, by means of the method according to the invention.

This pharmaceutical composition can be characterized in that it comprises:
from 50% to 70% by weight, preferably from 55% to 65% by weight, of moxifloxacin hydrochloride,
from 1% to 10%, preferably from 4% to 8% by weight, of a disintegrant,
from 1% to 6%, preferably from 2% to 4% by weight, of a binder,
from 10% to 30%, preferably from 18% to 25% by weight, of a diluent,
from 0.5% to 4%, preferably from 2.5% to 3.5% by weight, of a lubricant,
from 0.1% to 3%, preferably from 1% to 2% by weight, of a flow agent, and
from 1% to 5%, preferably from 2.5% to 3.5% of a coating agent.

The excipients and the moxifloxacin hydrochloride which are preferred correspond to those previously mentioned in the description of the method according to the invention (see in particular the section entitled “Excipients suitable for implementing the method according to the invention”.

An example of a composition according to the invention comprises:
from 55% to 65% by weight of moxifloxacin hydrochloride,
from 1% to 8% by weight of a disintegrant, preferably disodium sodium glycolate and/or sodium croscarmellose,
from 2% to 6% of a binder, preferably copovidone,
from 18% to 30% by weight of a diluent, preferably selected from microcrystalline cellulose, dibasic calcium phosphate and pregelatinized starch,
from 2.5% to 3.5% by weight of a lubricant, preferably magnesium stearate and/or talc,
from 1% to 2% by weight of a flow agent, preferably colloidal silica, and
from 1% to 5% of a coating agent, preferably based on polyvinyl alcohol, on HPMC or on povidone.

Another example of a composition according to the invention comprises:
from 55% to 65% by weight of moxifloxacin hydrochloride,
from 4% to 8% by weight of sodium croscarmellose as disintegrant,
from 2% to 4% by weight of copovidone as binder,
from 18% to 25% by weight of a mixture of microcrystalline cellulose and pregelatinized starch, as diluent,
from 2.5% to 3.5% of a mixture of magnesium stearate and talc, as lubricant, and
from 1% to 2% of colloidal silica as flow agent.

Said composition may comprise 2.5% to 3.5% of a coating agent based on polyvinyl alcohol, on HPMC or on povidone.

It goes without saying that the pharmaceutical composition according to the invention comprises a water content of less than 2% by weight, preferably less than 1.5% by weight, even more preferably less than 1% or even less than 0.5%. It also goes without saying that the moxifloxacin hydrochloride is essentially in anhydrous form.

The pharmaceutical composition according to the invention may be packaged in any type of packaging. For example, when the pharmaceutical composition according to the invention is in the form of tablets, these tablets may be packaged in bottles, or in blister packs, for example of Alu-Alu blister type. A subject of the invention is also a bottle or a blister pack containing tablets according to the invention.

The pharmaceutical composition according to the invention is intended for oral administration. It can be used in the treatment of infectious diseases in a subject. The infectious diseases of interest encompass certain respiratory infections (bronchitis, sinusitis, pneumonia) or gynaecological infections.

A subject of the invention is also a method for treating an infectious disease in a subject, comprising the administration of a therapeutically effective amount of the composition according to the invention, in particular of a pharmaceutical composition obtained by the method according to the invention.

The aim of the examples hereinafter is to illustrate the invention without, however, limiting the scope thereof.

### Example 1

### Study of the Stability of the Anhydrous Moxifloxacin Hydrochloride

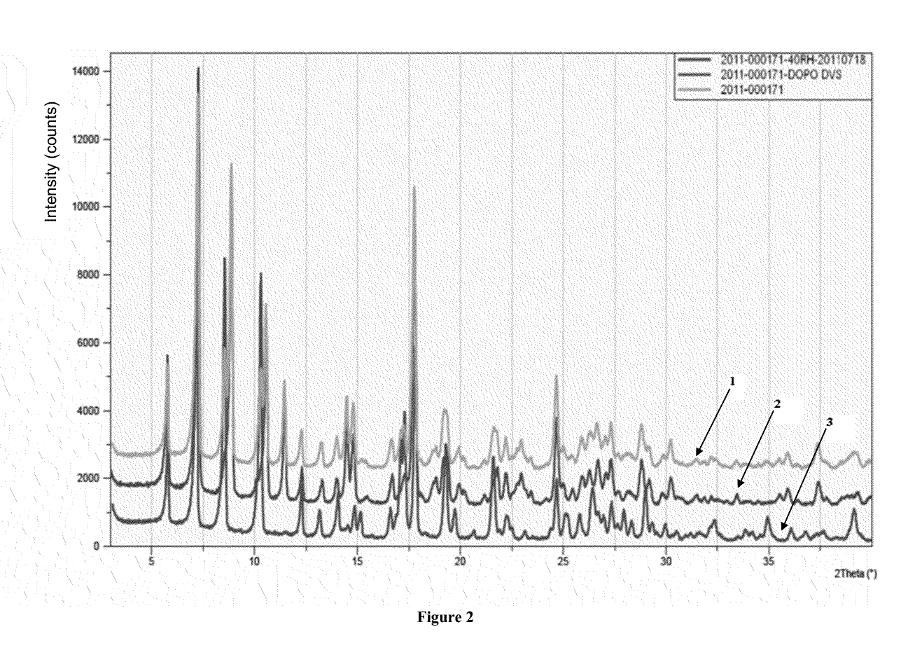

The X-ray powder diffraction spectrum of the anhydrous moxifloxacin hydrochloride is shown in FIG. 2 (see curve 1).

The anhydrous moxifloxacin hydrochloride is exposed for several hours to a relative humidity of approximately 40%, which causes an increase in its water content. At the end of the exposure, the water content of the active ingredient is approximately 4% by weight. As attested to by the XRPD spectrum (see curve 3, FIG. 2), the anhydrous moxifloxacin hydrochloride has been converted into a hydrate polymorphic form. Such results illustrate the low stability of the anhydrous moxifloxacin hydrochloride in the presence of a humidity level of 40%. The conversion of the anhydrous moxifloxacin hydrochloride into a hydrate form becomes significant for a water content above 2%.

The drying of the hydrate form obtained for several hours enables to decrease its water content to a value of less than 1% by weight, which results in its conversion into the starting anhydrous form (see curve 2, FIG. 2). This experiment demonstrates the possibility of removing the crystallization water molecules and of returning to the starting anhydrous polymorphic form by means of a simple heating step, despite the strong interactions that exist between the moxifloxacin molecules and the crystallization water molecules.

### Example 2

### Tablets According to the Invention and Production Method

A film-coated tablet according to the invention may have the following composition:

| | | | | % |
|---|---|---|---|---|
| | | | | (by weight |
| | | | | relative to |
| | | | Water | the total |
| | | | content | weight |
| | | | (% by | of the |
| Ingredient | Function | mg/tab | weight)* | tablet) |
| Moxifloxacin HCl* | active | 436.40 | <1% | 62.34 |
| (anhydrous form) | ingredient | | | |
| Sodium | disintegrant | 40.80 | <6% | 5.82 |
| croscarmellose | | | | |
| Copovidone | binder | 20.40 | <5% | 2.91 |
| Microcrystalline | diluent | 116.80 | <5% | 16.69 |
| cellulose | | | | |
| Pregelatinized corn | diluent | 35.00 | <7% | 5.00 |
| starch | | | | |
| Talc | lubricant | 10.20 | — | 1.46 |
| Colloidal silica | flow agent | 10.20 | — | 1.46 |
| Magnesium stearate | lubricant | 10.20 | — | 1.46 |

Coating

| | | | |
|---|---|---|---|
| Opadry II white ® | coating agent | 19.72 | 2.82 |
| Red iron oxide | colourant | 0.28 | 0.04 |

| | | |
|---|---|---|
| TOTAL | 700.00 | 100.000 |

*the amount of moxifloxacin hydrochloride contained in a tablet corresponds to a dose of 400 mg of moxifloxacin
*the water content can be determined using the Karl-Fisher titration method.

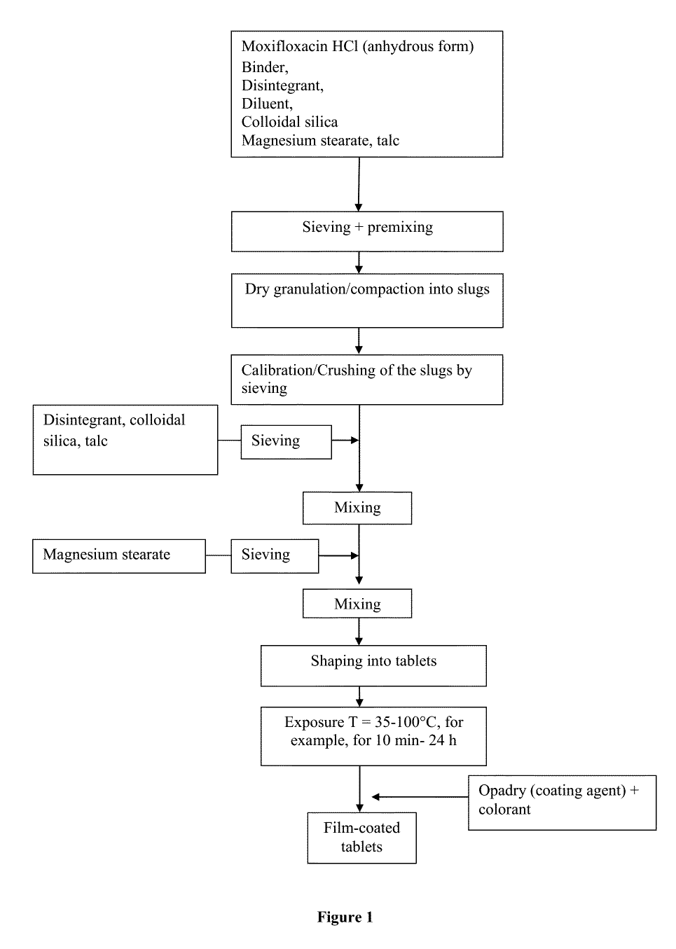

Such film-coated tablets can be prepared according to the method shown in FIG. 1. Briefly:
an anhydrous moxifloxacin hydrochloride with a water content of less than 1% is provided. For example, one may use of an anhydrous moxifloxacin hydrochloride which has a water content of 0.3% by weight,
the following excipients are provided: copovidone, sodium croscarmellose, microcrystalline cellulose, colloidal silica and magnesium stearate. If necessary, one or more of the excipients is subjected to a drying step in order to lower its water content,
the moxifloxacin hydrochloride and the excipients are sieved. The moxifloxacin hydrochloride is mixed with the microcrystalline cellulose, the copovidone and the pregelatinized starch and with a part of the sodium croscarmellose, of the colloidal silica, of the talc and of the magnesium stearate,
a dry granulation of the mixture obtained above is carried out (precompacting) so as to obtain slugs. A roll compactor can be used for this purpose,
the slugs are crushed through a sieve so as to obtain dry granules,
the dry granules are mixed with the remaining excipients (sodium croscarmellose, colloidal silica, talc and magnesium stearate),
the mixture of granules and excipients is compressed so as to obtain tablets,
the tablets formed are exposed to a temperature of 45° C. for 15-30 min, and
the tablets are coated with a coating agent of Opadry II combined with a colourant (red iron oxide).

The water content of the tablets is approximately 1.5% by weight.

The X-ray diffraction analysis of the starting moxifloxacin hydrochloride substance and of the final tablet shows the absence of conversion of the anhydrous moxifloxacine.HCl to other polymorphic forms during the formulating method.

Other examples of tablets according to the invention are:

Example of tablets according to the invention

| Ingredient | Function | mg/tab | % |
|---|---|---|---|
| Moxifloxacin HCl | active ingredient | 436.40 | 62.34 |
| (anhydrous form) | | | |
| Sodium croscarmellose | disintegrant | 20.40 | 2.91 |
| Copovidone | binder | 20.40 | 2.91 |
| Microcrystalline cellulose | diluent | 172.2 | 24.6 |
| Talc | lubricant | 10.20 | 1.46 |
| Colloidal silica | flow agent | 10.20 | 1.46 |
| Magnesium stearate | lubricant | 10.20 | 1.46 |

| | | |
|---|---|---|
| TOT | 680.00 | 97.14 |

Coating

| | | | |
|---|---|---|---|
| Opadry II white ® | coating agent | 19.72 | 2.82 |
| (based on PVA) | | | |
| Red iron oxide | colourant | 0.28 | 0.04 |

| | | |
|---|---|---|
| TOT | 700.00 | 100.00 |

Example of tablets according to the invention

| Ingredient | Function | mg/tab | % |
|---|---|---|---|
| Moxifloxacin HCl | active ingredient | 436.40 | 62.34 |
| (anhydrous form) | | | |
| Sodium starch glycolate | disintegrant | 20.40 | 2.91 |
| Copovidone | binder | 32.60 | 4.66 |
| Dibasic calcium | diluent | 135.00 | 19.28 |
| phosphate | | | |
| Pregelatinized starch | diluent | 25.00 | 3.57 |
| Talc | lubricant | 10.20 | 1.46 |
| Colloidal silica | flow agent | 10.20 | 1.46 |
| Magnesium stearate | lubricant | 10.20 | 1.46 |

| | | |
|---|---|---|
| TOT | 680.00 | 97.14 |

Coating

| | | | |
|---|---|---|---|
| Opadry white ® | coating agent | 19.72 | 2.82 |
| (based on HPMC) | | | |
| Red iron oxide | colourant | 0.28 | 0.04 |

| | | |
|---|---|---|
| TOT | 700.00 | 100.000 |

These tablets can be prepared according to the method shown in FIG. 1 and have, at the end, a water content of less than 2%.

### Example 3

### Stability Study

The film-coated tablets obtained according to Example 2 were packaged in Alu-Alu blisters and stored for 1 month at a temperature of 25° C. in the presence of a relative humidity (RH) of approximately 60%, or at a temperature of 40° C. and at an RH of approximately 75° C. At the end of this treatment, the tablets were analyzed by X-ray diffractometry. Their water content is also determined.

Results:

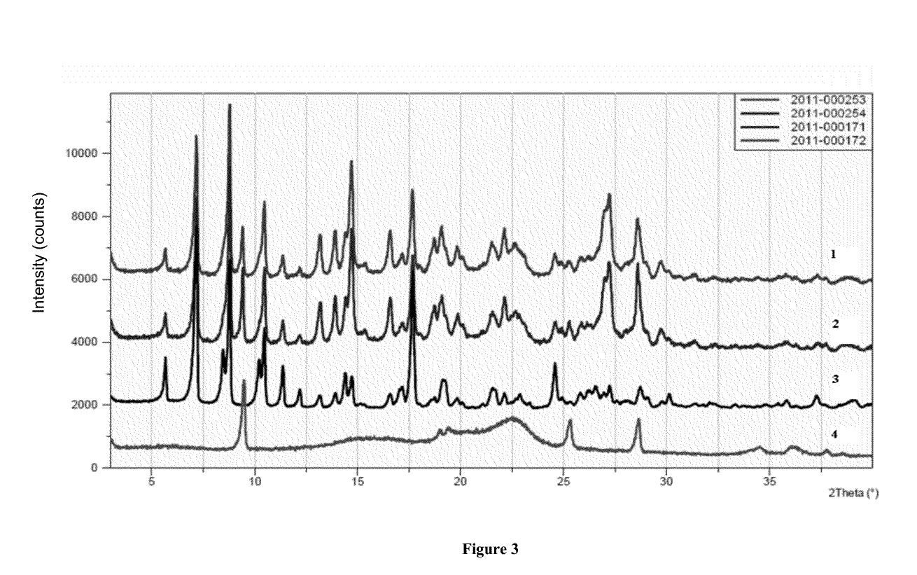

The X-ray diffraction spectra of the tablets after storage (see FIG. 3, curves 1 and 2) exhibit diffraction peaks similar to those of the spectrum of the anhydrous moxifloxacin hydrochloride powder (see FIG. 3, curve 3) and also some additional peaks that can be observed on the spectrum of the placebo tablet (FIG. 3, curve 4). There was therefore no significant conversion of the starting anhydrous moxifloxacin hydrochloride into a hydrate form during the storage of the tablets. Moreover, the water content of the tablets after storage is approximately 1.5% by weight. This attests to the stability of the tablets obtained according to the invention.

In conclusion, the water content of the tablets at the end of the manufacturing method must be less than 2%, preferably less than or equal to 1.5%, in order to avoid conversion of the anhydrous moxifloxacin hydrochloride into a hydrate form.

Additional stability experiments were carried out in order to confirm these results: tablets according to Example 2 were packaged either in Alu-Alu blister packs or in bottles. The bottles and the blister packs were stored for 6 months at a temperature of 40° C. in the presence of a relative humidity of approximately 75%. At the end of storage, the tablets were analyzed by X-ray diffractometry. Their water content was also determined After storage, the tablets have a water content of less than 2%. The X-ray diffractometry analyses show that there was no quantifiable conversion of the anhydrous moxifloxacin hydrochloride into a hydrate form during storage.

### Example 4

### Additional Test Regarding the Effect of the Heating Step d)

Tablets were prepared by means of the method described in Example 2, but without carrying out the heating stage and the film-coating step. The non-film-coated tablets thus obtained were exposed for several hours to a high level of humidity. At the end of exposure, the water content of the tablets is approximately 4% by weight and the ahydrous moxifloxacin hydrochloride has been partially converted into a hydrate form. A part of the tablets was placed at 100° C. for 1 h and another part at 80° C. for 2 h, in an oven.

In both cases, the heating step enabled to bring the water content of the tablets back to a value of less than 2% by weight. The X-ray powder diffraction analysis showed that the heating step enabled to reconvert the hydrates formed into the starting anhydrous form.

1-15. (canceled)

16. A method for preparing a pharmaceutical moxifloxacin hydrochloride composition in solid form, comprising the steps of:
a) providing moxifloxacin hydrochloride having a water content of less than 2% by weight,
b) mixing moxifloxacin hydrochloride of step a) with one or more pharmaceutically acceptable excipients,
c) shaping the mixture obtained in step b) so as to obtain a solid form,
d) subjecting the solid form obtained in step c) to a temperature ranging from 35° C. to 100° C., and
e) coating the solid form with a coating agent.

17. The method of claim 16, wherein moxifloxacin hydrochloride is in anhydrous form.

18. The method of claim 16, wherein the solid form obtained at the end of step d) has a water content of less than 2% by weight.

19. The method of claim 16, in which step e) is carried out directly at the end of step d).

20. The method of claim 16, wherein step d) is carried out at a temperature of 45° C. to 85° C. for a period ranging from 10 min to 24 h.

21. The method of claim 16, wherein in step b), moxifloxacin hydrochloride is mixed at least with a diluent and a binder.

22. The method according of claim 16, wherein in step b), moxifloxacin hydrochloride is also mixed with at least an excipient selected from the group consisting of a disintegrant, a lubricant, a flow agent, and mixtures thereof.

23. The method according of claim 16, wherein, in step c), the solid forms are obtained by direct compression or by dry granulation.

24. The method of claim 16, wherein step c) comprises the substeps of:
c1) carrying out a dry granulation of the mixture obtained in step b) so as to obtain granules and, optionally, sieving the granules obtained,
c2) mixing the granules obtained in substep c1) with one or more excipients, preferably selected from the group consisting of a disintegrant, a lubricant and a flow agent, and
c3) shaping the granules obtained at the end of substep c2) in the form of tablet(s).

25. The method of claim 24, wherein:
in step b), the moxifloxacin hydrochloride is mixed with a diluent, a disintegrant, a binder, a flow agent and a lubricant, and
in substep c2), the granules are mixed with a disintegrant, a lubricant and a flow agent.

26. A pharmaceutical moxifloxacin hydrochloride composition obtainable, or obtained, by the method as defined in claim 16.

27. A pharmaceutical moxifloxacin hydrochloride composition comprising:
from 50% to 70% by weight, preferably from 55% to 65% by weight, of moxifloxacin hydrochloride,
from 1% to 10% by weight, of a disintegrant,
from 1% to 6% by weight of a binder,
from 10% to 30% by weight of a diluent,
from 0.5% to 4% by weight of a lubricant,
from 0.1% to 3% of a flow agent, and
from 1% to 5% by weight of a coating agent,
the percentages being expressed relative to the total weight of the pharmaceutical composition, and wherein the water content is less than 2% by weight.

28. The pharmaceutical moxifloxacin hydrochloride composition of claim 27, wherein:
the disintegrant is selected from the group consisting of a sodium croscarmellose, a crospovidone, a hydroxypropyl cellulose which is optionally weakly crosslinked, a sodium starch glycolate, and mixtures thereof,
the binder is selected from the group consisting of a copovidone, a povidone, a hydroxypropyl cellulose, a hydroxypropylmethyl cellulose, and mixtures thereof,
the diluent is selected from the group consisting of a starch, a pregelatinized starch, a microcrystalline cellulose, a lactose monohydrate, a sodium phosphate, and mixtures thereof,
the lubricant is selected from the group consisting of stearate salts and mixtures thereof,
the flow agent is a silicon compound and
the coating agent is selected from coating agents based on polyvinyl alcohol, on HPMC or on povidone.

29. The pharmaceutical moxifloxacin hydrochloride composition according to claim 28, comprising:
from 55% to 65% by weight of moxifloxacin hydrochloride,
from 4% to 8% by weight of sodium croscarmellose as disintegrant,
from 2% to 4% by weight of copovidone as binder,
from 18% to 25% by weight of a mixture of microcrystalline cellulose and pregelatinized starch, as diluent,
from 2.5% to 3.5% of a mixture of magnesium stearate and talc, as lubricant, and
from 1% to 2% of colloidal silica as flow agent.

30. A method for treating an infectious disease in a subject, said method comprising administering the subject with the pharmaceutical composition of claim 26 or 27.
